(19) United States
(12) Patent Application Publication
(10) Pub. No.: US 2011/0270028 A1
(43) Pub. Date: Nov. 3, 2011

(54) BIOCOMPATIBLE AND BIOSTABLE IMPLANTABLE MEDICAL DEVICE

(75) Inventor: Babak Honaryar, Orinda, CA (US)

(73) Assignee: ALLERGAN, INC., Irvine, CA (US)

(21) Appl. No.: 13/113,907

(22) Filed: May 23, 2011

Related U.S. Application Data
(63) Continuation-in-part of application No. 12/887,730, filed on Sep. 22, 2010.
(60) Provisional application No. 61/330,266, filed on Apr. 30, 2010.

(51) Int. Cl. A61F 2/00; G01L 9/00

(52) U.S. Cl. 600/37; 73/753

(57) ABSTRACT

The present invention is related to a biocompatible and biostable implantable medical device. The present invention can include an implantable medical device including an electro-mechanical component. The electro-mechanical component can be coated with various novel and nonobvious coating combinations designed to promote biocompatibility and biostability.

### CROSS REFERENCE TO RELATED APPLICATIONS

This application is a continuation-in-part of and claims the benefit and priority of U.S. Non-Provisional patent application Ser. No. 12/887,730, entitled “BIOCOMPATIBLE AND BIOSTABLE IMPLANTABLE MEDICAL DEVICE” filed on Sep. 22, 2010, which in turn claims the benefit and priority of U.S. Provisional Application No. 61/330,266, entitled “BIOCOMPATIBLE AND BIODURABLE, ELECTRONICALLY ENHANCED ACCESS PORT FOR A FLUID FILLED IMPLANT” filed on Apr. 30, 2010. The entire disclosures of both of these applications are incorporated herein by reference.

### FIELD

The present invention broadly relates to medical devices and more specifically, to a biocompatible and biostable implantable medical device.

### BACKGROUND

There are numerous varieties of implantable medical devices, utilizing electronics or electronic-based enhancements. One such implantable medical device may be an adjustable gastric banding apparatus which provide an effective and substantially less invasive alternative to gastric bypass surgery and other conventional surgical weight loss procedures. Unlike gastric bypass procedures, the gastric band apparatus is reversible and require no permanent modification to the gastrointestinal tract.

Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND APO (Allergan, Inc., Irvine, Calif.) gastric band. Generally, gastric bands are placed about the cardia, or upper portion, of a patient's stomach forming a stoma that restricts the passage of food into a lower portion of the stomach. When the stoma is of an appropriate size that is restricted by a gastric band, food held in the upper portion of the stomach provides a feeling of satiety or fullness that discourages overeating. The adjustable gastric banding apparatus may include a port fitted with a pressure sensor which measures the pressure in the saline solution or a port that transmits a signal for easier detection of its location in the body, etc.

While beneficial, these medical devices come with challenges. For example, as these devices are to be implanted into a human body, it is important that these devices do not cause cytotoxicity and undesirably react with the surrounding body tissues. In other words, they should be “biocompatible.” Furthermore, these medical devices should be “biostable,” that is these medical devices should not be compromised by the interstitial body fluids and saline solution for a substantial period of time (e.g., five years or more).

Some attempts have been made to ensure that the medical device is properly implantable inside the human body. For example, Spehr (U.S. Pat. No. 6,240,320) discloses that biocompatible material such as diamond-like carbon, sapphire, parylene compounds, diamond, or like materials may be used to coat an exterior of the electrode member. However, Spehr suffers from, among other drawbacks, utilizing only one coating, and therefore does not address all of the essential requirements for a successful long-term function. Such requirements can include, for example, long-term biocompatibility (five years or more), the ability to coat relatively uniformly and thoroughly over an abrupt topology in a conformal manner, provide a significant barrier against water molecule penetration or transmission, utilize a deposition temperature and other processing parameters which are not too harsh for the substrate material and the electromechanical device being coated, non-conductivity of the portion of the coating that directly contacts an electrical equipment, cost-effective and the ability to stay attached to the substrate materials and retain its moisture barrier properties despite (i) abrasion caused by handling during assembly; (ii) thermal expansion and contraction during shipping and handling and then due to operation of the device after implantation; (iii) material aging; (iv) chemical interaction between adjacent materials; and (v) exposure to sterilization, such as heat, chemicals or radiation.

Adamis (U.S. Pat. No. 7,563,255) discloses coating devices contacting tissue or bio fluid with biocompatible material, such as, polyethyleneglycol, polyvinylchloride, polycarbonate, polysulfone, polytetrafluoroethylene, parylene, titanium or the like, prior to implantation. However, Adamis suffers from drawbacks, one of which includes depositing titanium on parylene, which is problematic as the deposition of titanium may require a very high temperature (e.g., greater than 140 degrees Celsius) thereby destroying the components it is intended to protect. In other words, many components would not be able to withstand the process of titanium deposition. In addition, Adamis ignores the issue of abrupt geometries (i.e., titanium deposition requires a relatively flat substrate surface). Furthermore, a conductivity of the titanium layer inhibits RF transmission and distorts magnetic coupling which may be necessary to energize the implanted electronics with no batteries.

### SUMMARY

In accordance with exemplary embodiments, the present invention provides for a biocompatible and biostable medical device that addresses the needs in the prior art.

In accordance with exemplary embodiments, the present invention provides for a medical device, such as a port configured to detect the pressure of a fluid within the implant. In accordance with other exemplary embodiments, the present invention provides for various novel and nonobvious coating combinations designed to promote biostability and biocompatibility of electro-mechanical components in the medical devices, including, but not limited to, those disclosed herein.

In one embodiment, three inner layers (e.g., Epoxy, Parylene, DLC) may all be moisture barriers. In one embodiment, DLC is the best barrier, followed by Parylene and then Epoxy. These three materials are also all relatively biocompatible, therefore they all improve the biocompatibility of the final assembly. Of course each has other unique characteristics, e.g., Epoxy's ability to encapsulate an abrupt geometry and Parylene's ability to fill in pin holes and DLC's superior barrier properties in addition to its hardness which offers scratch resistance during handling before the final silicone layer is applied. The final layer which is silicone might not be a good moisture barrier but was chosen for its superior biocompatibility and soft contact.

In one embodiment, the present invention is a pressure sensor for use with a medical implantable device, the pressure sensor comprising (1) a circuit board having circuit components mounted thereon, (2) a first layer of a first material encapsulating the circuit board and acting as a moisture barrier for the circuit board, the first layer for conforming to a surface topology of the circuit board to create an even surface, and for improving the biocompatibility of the circuit board, (3) a second layer of a second material, the second layer positioned on the first layer and acting as a moisture barrier for the first layer, the second layer for filling one or more holes of the first layer, (4) a third layer of a third material positioned on the second layer and acting as a moisture barrier for the second layer, and (5) a fourth layer of a fourth material positioned on the third layer, the fourth layer for improving the biocompatibility of the circuit board.

In one embodiment, the present invention is a method for protectively coating a medical device having an exposed circuit board, the medical device for implantation into the human body for the treatment of obesity or obesity-related diseases, the method comprising: (1) applying a first layer of a first material to fully encapsulate the circuit board, the first layer for conforming to a surface topology of the circuit board to create an even surface, and for improving the biocompatibility of the circuit board, (2) applying a second layer of a second material onto the first layer, the second layer for filling one or more holes of the first layer, and for preventing moisture from contacting the circuit board, (3) applying a third layer of a third material onto the second layer, the third layer for preventing moisture from contacting the second layer, and (4) applying a fourth layer of a fourth material onto the third layer, the fourth layer for improving the biocompatibility of the circuit board.

In one embodiment, the present invention is a gastric banding system for the treatment of obesity, the gastric banding system comprising: (1) a gastric band configured to be disposed about an esophageal-gastric junction of a patient, the gastric band including a ring coupled to an inflatable portion, (2) a tubing fluidly coupled to the inflatable portion at a first end of the tubing, the tubing for carrying fluid to inflate the inflatable portion of the gastric band and for carrying fluid from the inflatable portion to deflate the inflatable portion, and (3) an access port fluidly coupled to the tubing at a position located at a second end of the tubing, the access port including an electro-mechanical pressure sensor, the electro-mechanical pressure sensor including: (a) a circuit board having circuit components mounted thereon, (b) a first layer of a first material encapsulating the circuit board, the first layer for conforming to a surface topology of the circuit board to create an even surface, and for improving a biocompatibility of the access port and providing a barrier against moisture, (c) a second layer of a second material, the second layer positioned over the first layer, the second layer for filling one or more holes of the first layer, and for further preventing the moisture from contacting the circuit board, and for improving the biocompatibility of the access port, (d) a third layer of a third material positioned over the second layer, the third layer for preventing moisture from contacting the second layer, and for improving the biocompatibility of the access port, and (e) a fourth layer of a fourth material positioned over the third layer, the fourth layer for further improving the biocompatibility of the access port, and for providing a softer contact with the surrounding body tissue.

### BRIEF DESCRIPTION OF THE DRAWINGS

The exemplary embodiments of the present invention will be described in conjunction with the accompanying drawing FIGS. in which like numerals denote like elements and:

FIG. 1 illustrates a gastric banding system comprising a coated electro-mechanical component according to an embodiment of the present invention;

FIG. 2A illustrates a coated electro-mechanical component according to an embodiment of the present invention;

FIG. 2B illustrates a close up view of the coated electro-mechanical component of FIG. 2A with various layers according to an embodiment of the present invention; and

FIG. 2C illustrates a cross-sectional view of an electro-mechanical component of FIG. 2A coated with various layers according to an embodiment of the present invention.

### DETAILED DESCRIPTION

In accordance with exemplary embodiments, the present invention comprises a biocompatible and biostable medical device, such as an electro-mechanical component for an implanted gastric band.

The term biostable or biostability can mean, for example, that an implantable device or object is capable of being in contact with living tissues or organisms and still function within the expected performance parameters. In one embodiment, a biostable object or implanted device can still function within the expected performance parameters, for example, for 5 years, or even 10 years or more while being in contact with the living tissues or organisms.

The term biocompatible or biocompatibility can mean, for example, that the implantable device or object is capable of being in contact with living tissues or organisms without causing harm to the living tissue or the organism. For example, the coating combination may be biocompatible over an extended period of time, such as for 1, 2, 5, 10, 15, 20, or more years.

Persons skilled in the art will readily appreciate that various aspects of the invention may be realized by any number of methods and devices configured to perform the intended functions. Stated differently, other methods and devices may be incorporated herein to perform the intended functions. It should also be noted that the drawing FIGS. referred to herein are not all drawn to scale, but may be exaggerated to illustrate various aspects of the invention, and in that regard, the drawing FIGS. should not be construed as limiting. Finally, although the present invention may be described in connection with various medical principles and beliefs, the present invention should not be bound by theory.

At the outset, it should be noted that while the present invention will be described primarily with reference to a port coupled to a hydraulically adjustable gastric band for detecting a pressure of a fluid in the gastric band, persons skilled in the art will readily appreciate that the port is not necessary for detection of the pressure of the fluid. Furthermore, persons skilled in the art will readily appreciate that the present invention advantageously may be applied to any one of the numerous varieties of implantable medical devices such as an access port fitted with a pressure sensor which measures the pressure in saline, an access port that transmits an electrical signal for easier detection of its location in the body when implanted, a pump that controls the fluid volume in the gastric band, breast implants with pressure sensors and the like. Similarly, while the present invention will be described primarily with reference to fluid-filled surgical implants, persons skilled in the art will readily appreciate that the present invention advantageously may be applied to other medical devices, whether fluid-filled or not.

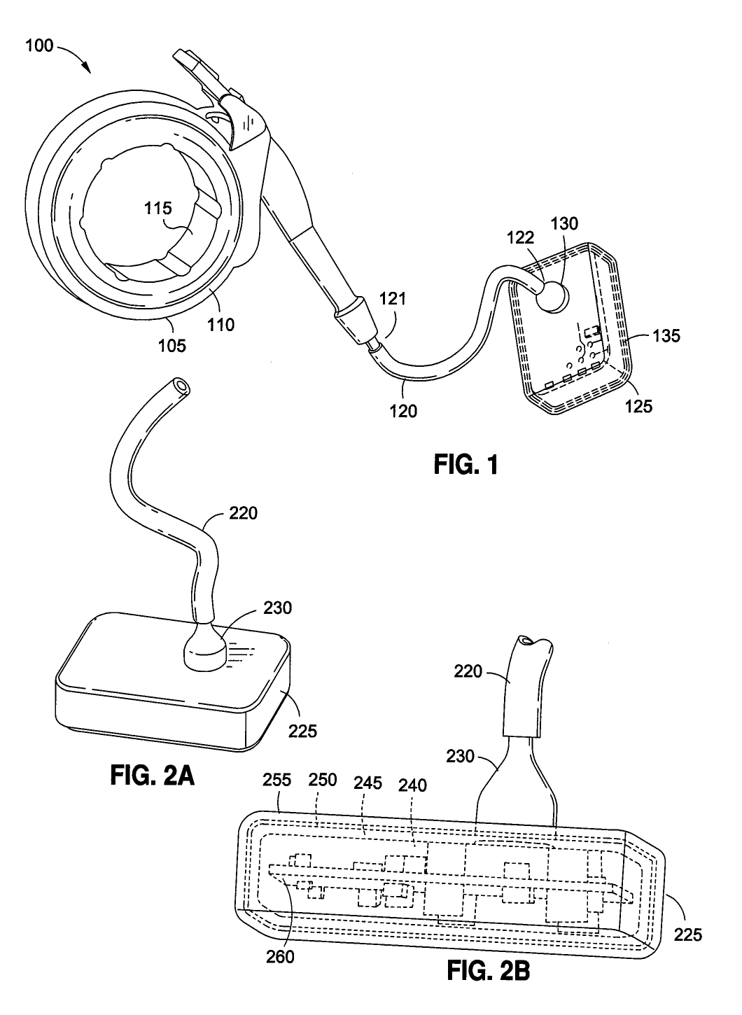

Turning to FIG. 1, a gastric banding system 100 is illustrated. The gastric banding system 100 may include a gastric band 105 having a ring 110 and an inflatable portion 115, the gastric band 105 being fluidly coupled to a port 125 via a tube 120 and a port interface 130. More particularly, the tube 120 may have a first end 121 coupled to the gastric band 105 and a second end 122 coupled to the port interface 130. The port 125 is shown to be pseudo-transparent to illustrate an embedded circuitry 135 which may be configured to monitor and report the pressure of the gastric band 105 through wireless communication (e.g., RF communication). In this manner, the port 125 may function as a pressure sensing device. As is well known in the art, the gastric banding system 100 may be implanted into the gastro-esophageal junction in a human body, where the inflatable portion 115 of the gastric band 105 is situated about a stomach region thereby controlling the size of the stoma, which in turn advantageously attempts to control the amount of food ingested by the patient.

The port 125 may also be implanted in the body and be configured to withstand the interstitial bodily fluids (i.e., biostable) while not causing cytotoxicity (i.e., biocompatible). Appropriate materials may be selected to achieve one or more of these goals. In one embodiment, the surface of the pressure sensing element, which may be in direct contact with the fluid which it is sensing, may be masked or isolated during the coating process.

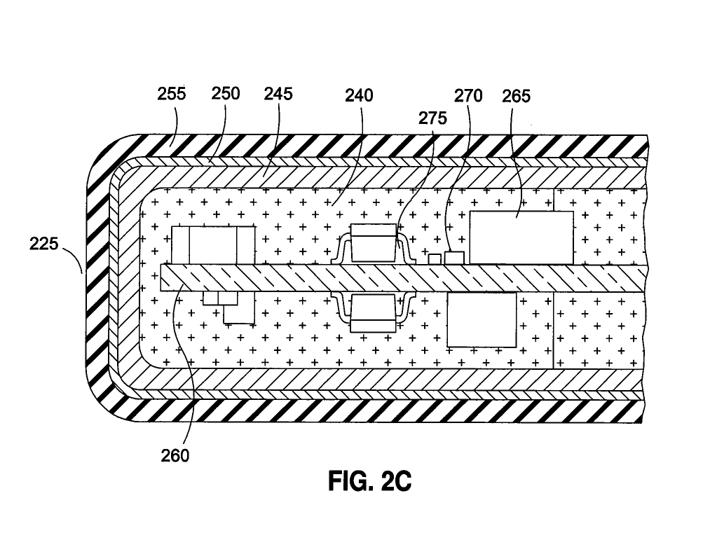

FIG. 2A is a close-up view of a port 225 having a port interface 230 connected to a tube 220. While the rest of the gastric banding system is not shown for simplicity, the tube 220 may be connected to a gastric band (e.g. the gastric band 105 of FIG. 1). FIG. 2A illustrates the port 225 after all coatings are applied. While the coating appears to be a box-shaped object with rounded corners, any shape is possible and may depend on the height/width and general topography of the underlying circuit board (not shown). Furthermore, while not discernible in FIG. 2A, the port 225 may be coated with multiple layers of different materials (as shown in FIG. 2B and/or FIG. 2C).

In accordance with exemplary embodiments, the present invention provides for coating combinations that isolate electro-mechanical components, including, but not limited to, printed circuit board assemblies, sensors, motors and other components typical to implantable medical devices, and/or components forming those objects listed above. The electro-mechanical components can be purely electrical components, purely mechanical components, or a hybrid of electrical and mechanical components.

Another exemplary coating combination may be able to coat relatively uniformly and/or thoroughly, over electro-mechanical components with an abrupt topology. Such electro-mechanical components can be objects with various abrupt geometries and/or various surface chemistries and thermal expansion properties such as a PCBA. Stated differently, an exemplary coating combination is capable of conformal coating.

Yet another exemplary coating combination may be a barrier against water molecule and other moisture penetration and/or transmission. Qualitatively, an exemplary coating combination may have a moisture vapor transmission rate (MVTR) roughly equivalent to that of titanium at approximately 25 μm (0.001 inches) thickness. MVTR is a measure of the passage of water vapor through a substance.

Exemplary coating combinations may remain attached to the substrate material and/or the electro-mechanical component being coated and retain its moisture barrier properties despite: (i) abrasion caused by handling during assembly; (ii) thermal expansion and contraction during shipping, handling, and operation of the electro-mechanical component after implantation; (iii) material aging; (iv) chemical interaction between adjacent materials; and (v) exposure to sterilization such as heat, chemicals or radiation.

The deposition temperature and other processing parameters of other exemplary coating combinations should not be too harsh for the substrate material and the electro-mechanical component being coated.

Depending on the electro-mechanical component being coated, yet other exemplary coating combinations may be non-conductive or conductive. For example, in one embodiment, the coating that directly contacts a PCA might not be conductive, and in certain embodiments, the subsequent over-layers may also be non-conductive (e.g., where the electro-mechanical components transmit or receive RF signals or is powered by an external magnetic field). Alternatively, in applications where a certain level of RFI shielding protects the device function, a conductive outer layer may be included.

In one embodiment, the coating combination, along with its coating process, may be reasonable in terms of cost, e.g., no more than the cost of the underlying electro-mechanical component being coated.

FIG. 2B illustrates four different layers forming the coating of the port 225, including a first layer 240 encapsulating a circuit board 260, a second layer 245 formed on the first layer 240, a third layer 250 formed on the second layer 245 and a fourth layer 255 formed on the third layer 250 in relationship to the port interface 230 and the tube 220. FIG. 2C illustrates a cross-sectional view clarifying the placement of the layers 240, 245, 250 and 255 with respect to a circuit board 260.

As shown in FIG. 2C, a first layer 240 may be designed to thoroughly encapsulate the underlying device (e.g., the circuit board 260). The first layer 240 may be a low viscosity Epoxy which encapsulates the underlying circuit board 260, resulting in an encapsulated object with flat outer surfaces. In this manner, the abrupt geometry of the underlying circuit board 260 (e.g., the different heights, widths, topography of the circuit components) are evened out by the Epoxy material, thereby forming a flat surface for a second layer 245. More particularly, the Epoxy may create a flat surface conforming the height and width of each component (e.g., components 265 and 270 having different heights, widths, shapes, etc. are now “evened” out). Furthermore, the Epoxy may fill in any gap (e.g., gap 275) within elements or between elements.

The amount of Epoxy applied may be the minimum to ensure that the height of the tallest element is covered while spanning the entire length and width of the circuit board 260. By overmolding or encapsulating the Epoxy to form a flat and/or even outer surface about the perimeter of the circuit board, the Epoxy transforms an uneven topography formed by various circuit elements into an object with substantially flat surfaces, which advantageously increases the likelihood of a uniform and contiguous second layer 245.

In addition, the Epoxy possesses a relatively high service temperature, which allows for application of high temperature coatings. Furthermore, Epoxy, such as USP class VI rated Epo-Tek 354, reduces concerns over biocompatibility of the resulting coated port 225.

A second layer 245 may be deposited, applied or positioned on the first layer 240. In one embodiment, the second layer 245 may be Parylene. Parylene is a polymeric conformal coating applied through a chemical vapor deposition process (CVD) and may be offered in various chemistries with different properties. For instance, Parylene P is a Parylene variation with high penetration properties. However, Parylene P may not necessarily be optimized for moisture barrier properties. In one embodiment, Parylene P can be, for example, Parylene HT produced by Specialty Coating Systems or Parylene diX N produced by Kisco Conformal Coating. Parylene M is another variation of Parylene and may have improved moisture barrier properties. However, Parylene M may not have the penetrative properties of Parylene P. In one embodiment, Parylene M can be, for example, Parylene C produced by Specialty Coating Systems or Parylene diX D produced by Kisco Conformal Coating.

In one embodiment, the second layer 245 may be Parylene P, as Parylene P has a property of higher service temperature and therefore may be able to better withstand the processing temperature of the third layer 250 and the heat sterilization temperature.

The third layer 250 may be deposited, applied or positioned on the second layer 245. The third layer 250 may be a diamond-like carbon (DLC). DLC may have, among other features, excellent moisture barrier properties at very low thicknesses (e.g., as low as three p-inches). DLC may be very durable and is applied with a CVD, a plasma-enhanced CVD (PECVD), a physical vapor deposition (PVD) or variations thereof. By utilizing a DLC which may be applied at relatively low temperatures (e.g., under 130 degrees Celsius), the underlying circuit board 260 and/or the other layers (e.g., the layers 240 and 245) may be protected from damage caused by high temperatures (as these components may be susceptible to damage at temperatures higher than 130 degrees Celsius). DLC may be from Richter Precision, Inc. or Northeast Coating Technologies, among others.

The fourth layer 255 may be deposited, applied or positioned onto the third layer 250. The fourth layer may be an outer layer which directly contacts the bodily tissue upon implantation. In one embodiment, the fourth layer 255 may be constructed out of silicone rubber, through a dipping or overmolding process. The silicone rubber has proven to have excellent biocompatibility properties due to its customizable texture and porosity. By utilizing it as the fourth layer 255, the overall biocompatibility of the port 225 may be enhanced. Furthermore, the silicone rubber may have softer tissue contact and may further protect the underlying layers 250, 245 and 240 from abrasions and small impacts caused, for example, by handling during manufacturing. Some silicon rubber grades that may be used include MED-4850 from Nusil Corporation or Silastic Q7-4850 from Dow Chemical Corporation.

The materials of one embodiment of the layers 240, 245, 250 and 255 having been described, attention will now be turned to the thickness of each layer. As shown in FIG. 2C, the layers 240, 245, 250 and 255 might not be shown to scale.

In one embodiment, the inner or first layer 240 formed of Epoxy may encapsulate the entirety of the circuit board 260 and related components (e.g., components 265 and 270), and may be designed to create a surface of at least about 0.020 inches over the highest component thereby ensuring that every component is encapsulated.

The second layer 245 may be constructed out of Parylene P and may have a thickness in the range of about 0.004 to about 0.008 inches. Such a coating may prevent moisture from reaching the first layer 240 and also may penetrate the first layer 240 to fill in any potential pin holes, and further even out the surface formed by the first layer 240.

The third layer 250 may be constructed out of DLC and may be 4 μ-inches or thicker in one embodiment, but preferably at a thickness of about 40 μ-inches.

The fourth layer 255 may be a silicone rubber and may be constructed to have a thickness of about 0.02 to about 0.06 inches, providing excellent biocompatibility properties for the implanted port (e.g., port 125).

In one embodiment, three inner layers (e.g., Epoxy, Parylene, DLC) may all be moisture barriers. DLC is the best barrier, followed by Parylene and then Epoxy. These three materials are also all relatively biocompatible, therefore they all improve the biocompatibility of the final assembly. Of course, each has other unique characteristics, e.g., Epoxy's ability to encapsulate an abrupt geometry and Parylene's ability to fill in pin holes and DLC's superior barrier properties in addition to its hardness which offers scratch resistance during handling before the final silicone layer is applied. The final layer which is silicone might not be a good moisture barrier but may be chosen for its superior biocompatibility and soft contact.

The materials and properties of the different layers of the coating of the port 225 having been discussed, attention will be turned to the method of manufacturing. In one embodiment, the coating of the port 225 may be performed via the following method. First, a first layer of a first material (e.g., epoxy as described above) may be applied to fully encapsulate the circuit board, the first layer for conforming to a surface topology of the circuit board to create an even surface, and for improving the biocompatibility of the circuit board. Second, a second layer of a second material (e.g., parylene as described above) may be applied to the first layer, the second layer for filling one or more holes of the first layer, and for preventing moisture from contacting the circuit board. Next, a third layer of a third material (e.g., DLC as described above) may be applied onto the second layer, the third layer for preventing moisture from contacting the second layer. Then, a fourth layer of a fourth material (e.g., silicone rubber, as described above) may be applied onto the third layer thereby completing the coating process, the fourth layer for improving the biocompatibility of the circuit board.

In one embodiment, the one or more coatings or layers may be applied to various implantable medical devices such as an access port, a breast implant, a cardiac rhythm management device, a pacemaker, a cardioverter, a defibrillator, a neurostimulator, an activity sensor, a pressure sensor, a multi-sensor device, a drug delivery pump or device, a heart monitor, a respiratory monitor, an artificial kidney or other artificial organs aside from the heart, orthopedic implants with electronics incorporating stress, pressure or force sensors. In one embodiment, the various implantable medical devices are medical devices which may come in contact with interstitial body fluids, but do not come in contact with blood.

The foregoing disclosure is illustrative of the present invention and is not to be construed as limiting the invention. Although one or more embodiments of the invention have been described, persons skilled in the art will readily appreciate that numerous modifications could be made without departing from the spirit and scope of the present invention. For example, the above description of the thicknesses of the different layers of coatings is of one embodiment and is not intended to be limiting. Those of ordinary skill in the art will recognize that other thicknesses may be possible and is within the scope of the invention.

By way of mere example, persons skilled in the art will readily appreciate that the novel and nonobvious coating combinations designed to promote biostability described herein advantageously may be applied not just to surgical implants, but to any device or device component having biostability as a design requirement. In sum, it should be understood that all such modifications are intended to be included within the scope of the invention.

The terms “a,” “an,” “the,” and similar referents used in the context of describing the present invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

What is claimed is:

1. A pressure sensor for use with a medical implantable device, the pressure sensor comprising:
a circuit board having circuit components mounted thereon;
a first layer of a first material encapsulating the circuit board and acting as a moisture barrier for the circuit board, the first layer for conforming to a surface topology of the circuit board to create an even surface, and for improving the biocompatibility of the circuit board;
a second layer of a second material, the second layer positioned on the first layer and acting as a moisture barrier for the first layer, the second layer for filling one or more holes of the first layer;
a third layer of a third material positioned on the second layer and acting as a moisture barrier for the second layer; and
a fourth layer of a fourth material positioned on the third layer, the fourth layer for improving the biocompatibility of the circuit board.

2. The pressure sensor of claim 1 wherein the first material is Epoxy.

3. The pressure sensor of claim 2 wherein an outer surface of the first layer is at least about 0.020 inches apart from a highest component of the circuit components mounted thereon the circuit board.

4. The pressure sensor of claim 3 wherein the second material is parylene.

5. The pressure sensor of claim 4 wherein the second layer has a thickness in the range of about 0.004 to about 0.008 inches.

6. The pressure sensor of claim 5 wherein the third material is a diamond-like carbon (DLC).

7. The pressure sensor of claim 6 wherein the third layer has a thickness of at least about 4 μ-inches.

8. The pressure sensor of claim 7 wherein the fourth material is silicone rubber.

9. The pressure sensor of claim 8 wherein the fourth layer has a thickness in the range of about 0.02 to about 0.06 inches.

10. A gastric banding system for the treatment of obesity, the gastric banding system comprising:
a gastric band configured to be disposed about an esophageal-gastric junction of a patient, the gastric band including a ring coupled to an inflatable portion;
a tubing fluidly coupled to the inflatable portion at a first end of the tubing, the tubing for carrying fluid to inflate the inflatable portion of the gastric band and for carrying fluid from the inflatable portion to deflate the inflatable portion; and
an access port fluidly coupled to the tubing at a position located at a second end of the tubing, the access port including a electro-mechanical pressure sensor, the electro-mechanical pressure sensor including:
a circuit board having circuit components mounted thereon;
a first layer of a first material encapsulating the circuit board, the first layer for conforming to a surface topology of the circuit board to create an even surface, and for improving a biocompatibility of the access port and providing a barrier against moisture,
a second layer of a second material, the second layer positioned over the first layer, the second layer for filling one or more holes of the first layer, and for further preventing the moisture from contacting the circuit board, and for improving the biocompatibility of the access port,
a third layer of a third material positioned over the second layer, the third layer for preventing moisture from contacting the second layer, and for improving the biocompatibility of the access port, and
a fourth layer of a fourth material positioned over the third layer, the fourth layer for further improving the biocompatibility of the access port, and for providing a softer contact with surrounding body tissue.

11. The gastric banding system of claim 10 wherein the first material is Epoxy, and wherein an outer surface of the first layer is at least about 0.020 inches apart from a highest component of the circuit components mounted thereon the circuit board.

12. The gastric banding system of claim 11 wherein the second material is parylene, and wherein the second layer has a thickness in the range of about 0.004 to about 0.008 inches.

13. The gastric banding system of claim 12 wherein the third material is a diamond-like carbon (DLC), and wherein the third layer has a thickness of at least about 4 μ-inches.

14. The gastric banding system of claim 13 wherein the fourth material is silicone rubber, and wherein the fourth layer has a thickness in the range of about 0.02 to about 0.06 inches.

15. A method for protectively coating a medical device having an exposed circuit board, the medical device for implantation into the human body for the treatment of obesity or obesity-related diseases, the method comprising:
applying a first layer of a first material to fully encapsulate the circuit board, the first layer for conforming to a surface topology of the circuit board to create an even surface, and for improving the biocompatibility of the circuit board;
applying a second layer of a second material onto the first layer, the second layer for filling one or more holes of the first layer, and for preventing moisture from contacting the circuit board;
applying a third layer of a third material onto the second layer, the third layer for preventing moisture from contacting the second layer; and
applying a fourth layer of a fourth material onto the third layer, the fourth layer for improving the biocompatibility of the circuit board.

16. The method of claim 15 wherein the first material is Epoxy, and wherein an outer surface of the applied first layer is at least about 0.020 inches apart from a highest component of the circuit components mounted thereon the circuit board.

17. The method of claim 16 wherein the second material is parylene, and wherein the applied second layer has a thickness in the range of about 0.004 to about 0.008 inches.

18. The method of claim 17 wherein the third material is a diamond-like carbon (DLC), and wherein the applied third layer has a thickness of at least about 4 μ-inches.

19. The method of claim 18 wherein the fourth material is silicone rubber, and wherein the applied fourth layer has a thickness of at least about 0.02 inches.

20. The method of claim 18 wherein the fourth material is silicone rubber, and wherein the applied fourth layer has a thickness in the range of about 0.02 to about 0.06 inches.
